(19) United States
(12) Patent Application Publication
(10) Pub. No.: US 2011/0054533 A1
(43) Pub. Date: Mar. 3, 2011

(54) SPINOUS FUSION DEVICE

(76) Inventors: Lawrence J. Binder, Miami, FL (US); Stewart Eidelson, Boca Raton, FL (US)

(21) Appl. No.: 12/868,189

(22) Filed: Aug. 25, 2010

Related U.S. Application Data
(60) Provisional application No. 61/237,280, filed on Aug. 26, 2009.

(51) Int. Cl. A61B 17/70

(52) U.S. Cl. 606/249

(57) ABSTRACT

An implant device has a main body that has a top surface and a bottom surface. The implant device further has a first notch formed in the top surface for receiving a first spinous process and a second notch formed in the bottom surface for receiving a second spinous process.

### CROSS REFERENCE TO RELATED APPLICATIONS

This application claims priority to U.S. Provisional Application No. 61/237,280, entitled “Spinous Fusion Device Made From Human Femur,” and filed on Aug. 26, 2009, which is incorporated herein by reference.

### BACKGROUND

The central nervous system is part of an overall system that functions to coordinate human activity. It is made up of the brain and the spinal cord. The main function of the spinal cord is to act as a conduit to communicate neuronal signals from the brain to the rest of the body. Protecting the spinal cord is the spinal column, or what is commonly referred to as the spine or vertebral column. Anatomically, the spinal column is made up of several regions, including the cervical, thoracic, lumbar and sacral regions. The cervical spine is made up of seven vertebrae, and the cervical spine functions to support the weight of the head. The thoracic spine is made up of twelve vertebrae, and the thoracic spine functions to protect the organs located within the chest. Five vertebrae make up the lumbar spine. The lumbar spine contains the largest vertebrae, and the lumbar spine functions as the main weight bearing portion of the spine. Located at the base of the spine are the five fused vertebrae known as the sacrum. The coccyx sits at the base of the spinal column and consists of four fused vertebrae.

Each of the vertebrae associated with the various spinal cord regions is made up of a vertebral body, a posterior arch, and transverse processes. The vertebral body, often described as having a drum-like shape, is designed to bear weight and withstand compression or loading. In between the vertebral bodies are intervertebral discs. The discs help cushion the spine against various movements and can be the source of various diseases. The posterior arch of the vertebrae is made up of the lamina, pedicles, and facet joints. Transverse processes extend outwardly from the vertebrae and provide the means for muscle and ligament attachment, which aid in movement and stabilization of the vertebra.

One of the more common ailments associated with the spinal cord is damage to the spinal discs. Damage to the discs results from physical injury, disease, genetic disposition, or as part of the natural aging process. Disc damage often results in intervertebral spacing not being maintained, causing pinching of exiting nerve roots between the discs, resulting in pain. For example, disc herniation is a condition in which the disc material bulges from the disc space between the two vertebrae bodies. It is the bulging of the disc material which causes impingement on the nerves, manifesting in pain to the patient. In severe cases or in cases which have developed into spinal instability, the damaged disc material between the vertebral bodies is removed and replaced with spinal stabilization implants.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the figures.

FIG. 1 is a frontal view of a human skeleton.

FIG. 2 is a side view of a femur section cut away from the femur of the human skeleton depicted in FIG. 1.

FIG. 3 is a cross-sectional view of the human femur shaft depicted in FIG. 2.

FIG. 4 is a perspective view of the femur section depicted in FIG. 2.

FIG. 5 is a side view of the femur section depicted in FIG. 2 showing the shape of an exemplary implant device projected on the outer surface in accordance with an embodiment of the present disclosure.

FIG. 6 is a perspective view of the femur section of FIG. 2 showing the shape of the implant device projected on the outer surface.

FIG. 7 is a side view of the femur section of FIG. 2 showing the shape of another exemplary implant device projected on the outer surface in accordance with an embodiment of the present disclosure.

FIG. 8 is a cross-sectional view of the femur section of FIG. 2 taken along line B-B showing the hatched region that remains once the unnecessary portions of the femur are cut away from the implant device.

FIG. 9 is a cross-sectional view of the femur section of FIG. 2 taken along line B-B showing the hatched region that is to be cut away from the implant device.

FIG. 10 is a front view of an exemplary implant device that results from the cutting away of the unnecessary portions of the femur.

FIG. 11 is a cross sectional view of the implant device, such as is depicted in

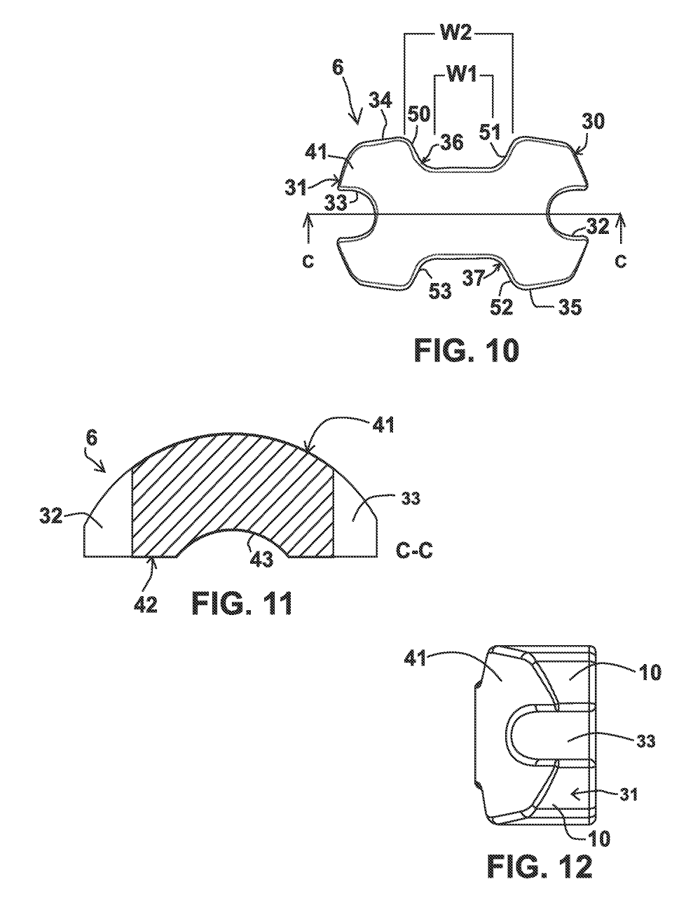

FIG. 10, taken along line C-C.

FIG. 12 is a side view of the implant device, such as is depicted in FIG. 10.

FIG. 13 is a front perspective view of the implant device, such as is depicted in FIG. 10.

FIG. 14 is a back perspective view of the implant device, such as is depicted in

FIG. 10.

FIG. 15 is a lateral view of a functional spinal unit.

FIG. 16 is a posterior view of the functional spinal unit, such as is depicted in FIG. 15.

FIG. 17 is a lateral view of the functional spinal unit, such as is depicted in FIG. 15 having the implant device, such as is depicted in FIG. 10, inserted within the functional spinal unit.

FIG. 18 is a posterior view of the functional spinal unit, such as is depicted in FIG. 16, having the implant device, such as is depicted in FIG. 10, inserted within the functional spinal unit.

FIG. 19 is a perspective view of the functional spinal unit, such as is depicted in FIG. 15.

FIG. 20 is a perspective view of the functional spinal unit, such as is depicted in FIG. 15, having the implant device, such as is depicted in FIG. 10, inserted within the functional spinal unit.

FIG. 21 is a flowchart depicting a method of making the spinal implant device, such as is depicted in FIG. 13.

### DETAILED DESCRIPTION

An implant device in accordance with an embodiment of the present disclosure is designed for insertion between bone structures. In particular, the implant device is designed for insertion between two spinous processes. The implant device prevents or limits the motion of the vertebrae.

In one embodiment, the implant device is made from a femur section of a skeleton. In this regard, a femur section is cut from the femur of a skeleton, and portions of the femur section are cut away leaving only the implant device.

The implant device is arch-shaped and has at least two opposing notches on a top side and a bottom side for mating with the spinous process. In one embodiment, the implant device further has two opposing notches on the sides for receiving an insertion instrument when the implant device is being inserted.

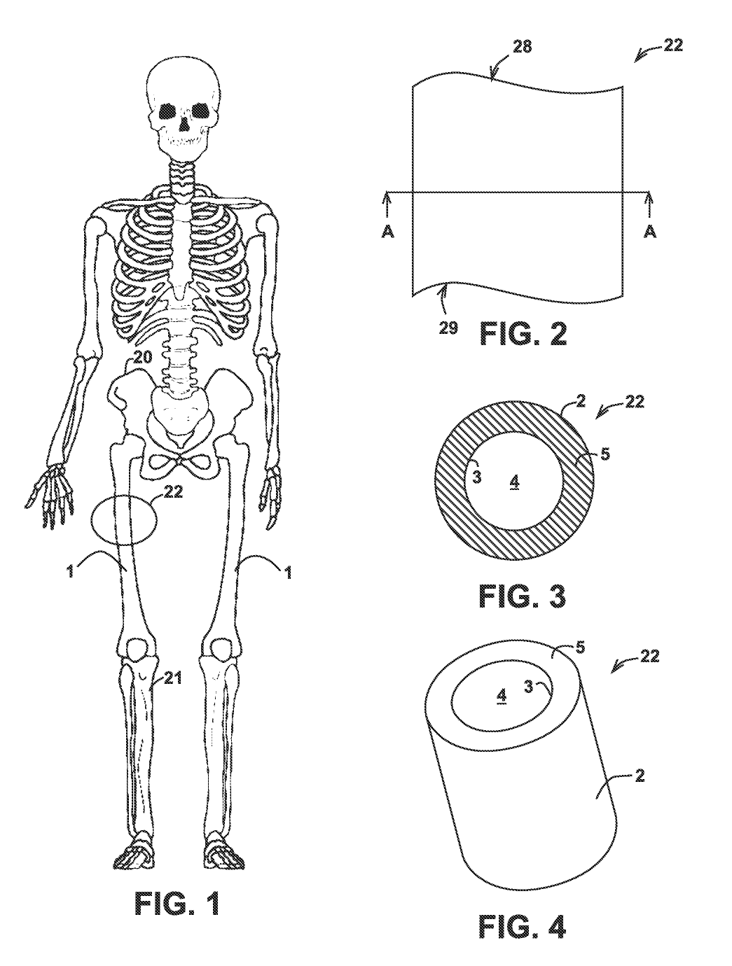

FIG. 1 is a frontal view of a human skeleton. The human skeleton has a femur 1 that is located between a pelvic girdle 20 and a tibia 21. In accordance with an embodiment of the present disclosure, a femur section 22 is cut from the femur 1.

FIG. 2 is a side view of the femur section 22 that is cut from the femur 1 of the human skeleton depicted in FIG. 1. Note that the top and bottom sides 28 and 29, respectively, are machined when the cut is made in the femur 1.

FIG. 3 is a cross-sectional view of the femur 1 taken along line A-A of FIG. 2. The femur section 22 has an intermedullary canal 4, which is a hollow opening that extends through the femur section 22. In addition, the femur section 22 has an outer wall 2, an inner wall 3, and a machined surface 5. Note that FIG. 3 shows only one view taken along A-A. However, a view in the opposing direction is substantially similar.

FIG. 4 is a perspective view of the femur section 22. As indicated hereinabove, the femur section 22 has an intermedullary canal 4, which is a hollow opening that extends through the femur section 22. In addition, the femur section 22 has the outer wall 2, the inner wall 3, and the machined surface 5.

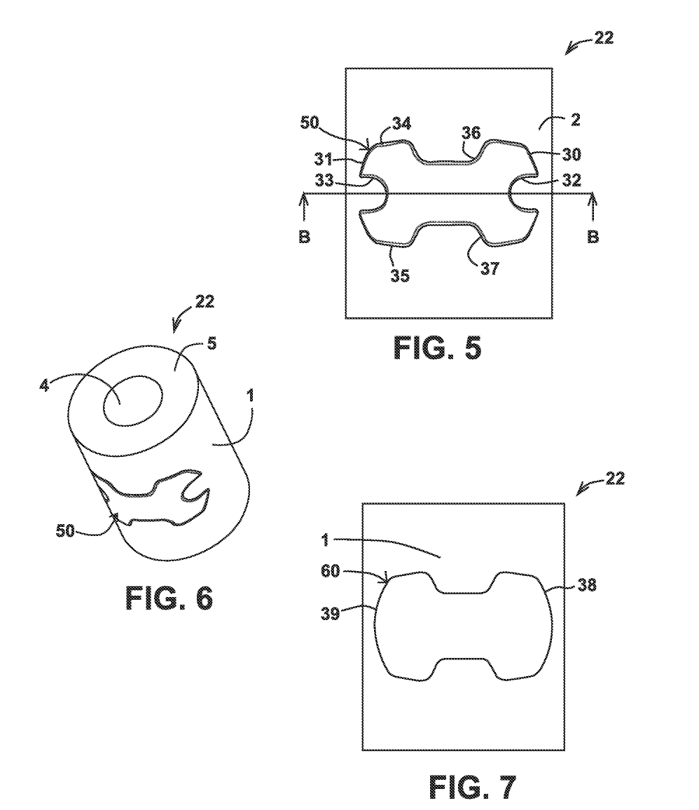

In the process of making an exemplary implant device (not shown) in accordance with an embodiment of the present disclosure, unneeded portions of the femur section 22 are cut away leaving only the implant device. In this regard, FIG. 5 is a front view showing the location and orientation of a projection onto the outer surface 2 of an outline 50 of the implant device that is to be cut away from the femur section 22. Further, FIG. 6 is a perspective view showing the location and orientation of the projection of the outline 50 onto the outer surface 2 of the implant device that is to be cut away from the femur section 22.

With reference to FIG. 5, the projection depicts a top side 34 of the implant device having a notch 36 therein. In addition, the projection depicts a bottom side 35 of the implant device having a notch 37 therein. Further, sides 30 and 31 also having notches 32 and 33, respectively, therein.

FIG. 7 is a front view showing the location and orientation of a different projection onto the outer surface 2 of an outline 60 of another exemplary implant device that is to be cut away from the femur section 22. The projection of the outline 60 in FIG. 7 differs from the projection in FIG. 5 in that there are no notches located on the sides 38 and 39 of the projection.

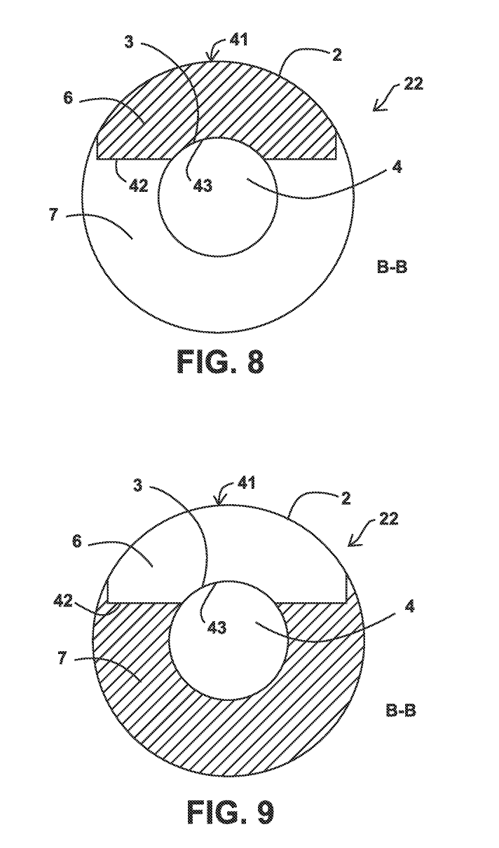

FIG. 8 shows a cross-sectional view of the femur section 22 taken along line B-B in FIG. 5. The cross-sectional view shows an exemplary implant device 6 (shown in the hatched section) that remains after the unneeded portion 7 of the femur section 22 is cut away. The implant device 6 has a front surface 41 that is made up of the outer wall 2 of the femur section 22. In addition, the implant device 6 has a back surface 42 that is partially made up of the inner wall 3. The intermedullary canal 4 creates an arch-shaped portion 43 on the back surface 42. FIG. 9 shows the cross-sectional view of the femur section 22 with the unneeded portion 7 (shown in the hatched section) that is to be removed.

Note that as described herein, the implant device 6 is made of human allograft. However, the implant device 6 may be made of other materials known in the art or future-developed. For example, the implant device 6 may be made from a synthetic implant grade polymer, such as PEEK or Polyethylene

FIG. 10 is a front view of the device 6. The device 6 comprises the top side 34 that has the notch 36 formed therein, and the device 6 comprises the bottom side 35 that has the notch 37 formed therein. The notches 36 and 37 in the top and bottom sides 36 and 37, respectively, are formed therein for receiving spinous processes, which is described herein with reference to FIG. 18.

The notch 36 has tapered walls 50 and 51 for receiving a spinous process, and the notch 37 has tapered walls 52 and 53 for receiving another spinous process. In one embodiment, the walls 50 and 51 have width w₁ at the bottom of the notch 36 that increases to width w₂ at the top of the notch 36. Further, the walls 52 and 53 are dimensioned substantially similar to the walls 50 and 51. In another embodiment, the walls 50 and 51 have a width w₁ at the bottom of the notch 36 that decreases to width w₂ at the top of the notch 36. In such an embodiment, the walls 52 and 53 are dimensioned substantially similar to the walls 50 and 51.

In addition, the device 6 comprises the sides 30 and 31. In one embodiment, the side 30 has the notch 32 formed therein, and the side 31 has the notch 33 formed therein. Notably, such notches 32 and 33 are for receiving an insertion tool when the device 6 is being implanted within a functional spinal unit, which is described further herein with reference to FIGS. 15-20. Note that the notches 32 and 33 are not necessary in other embodiments of the present disclosure, such as the embodiment depicted in FIG. 7.

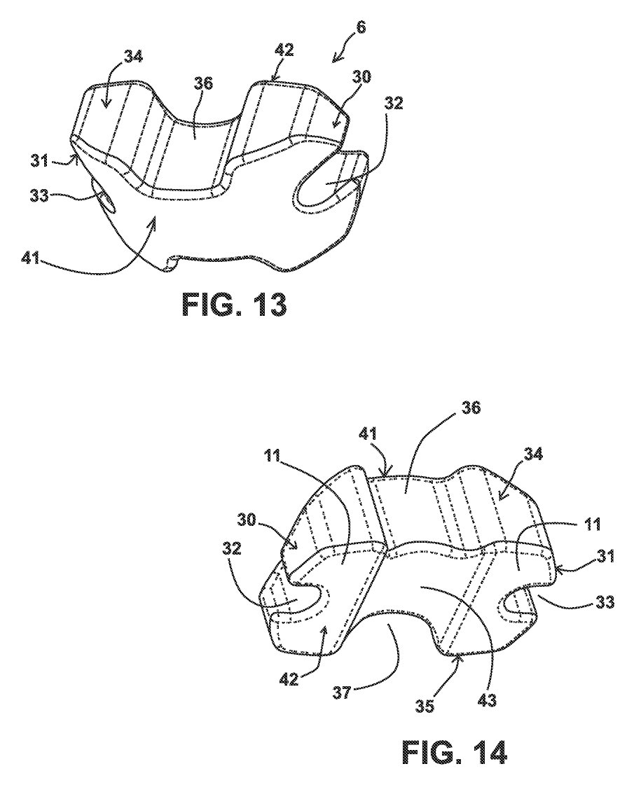

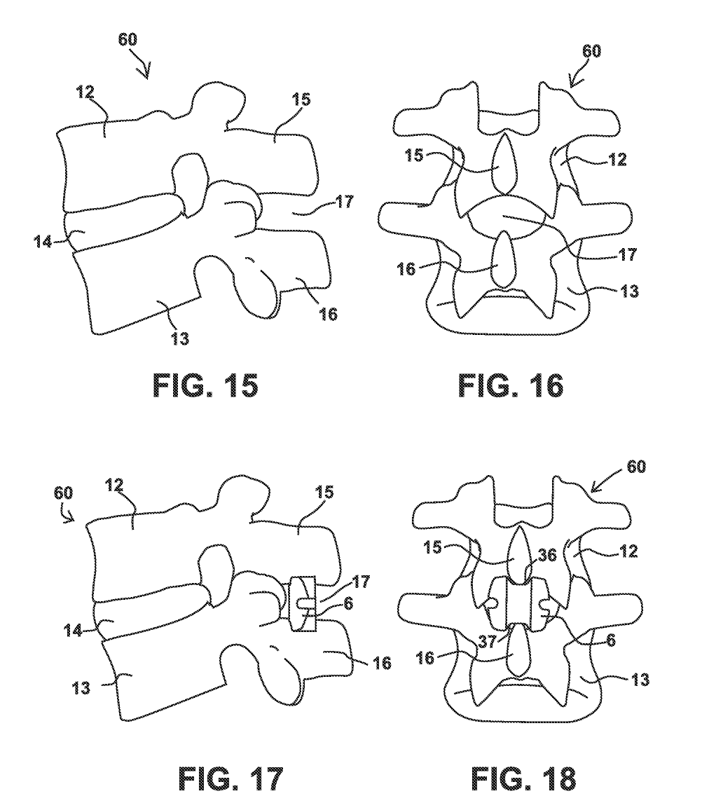

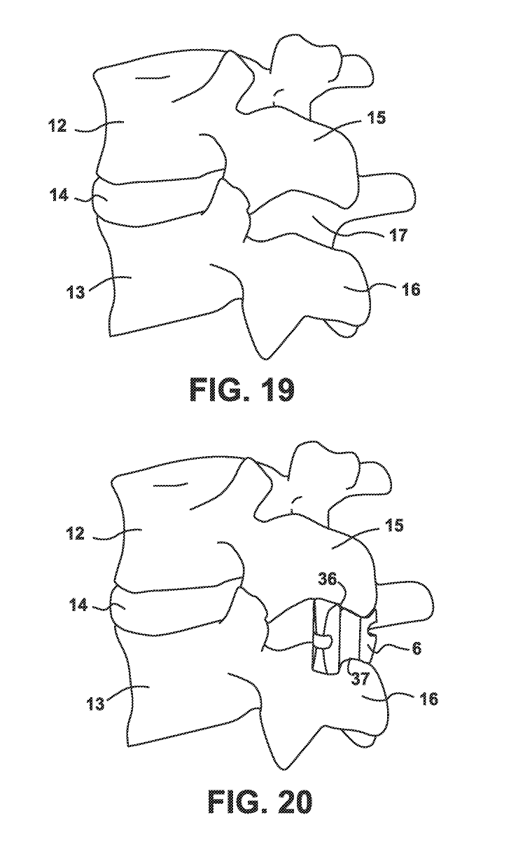

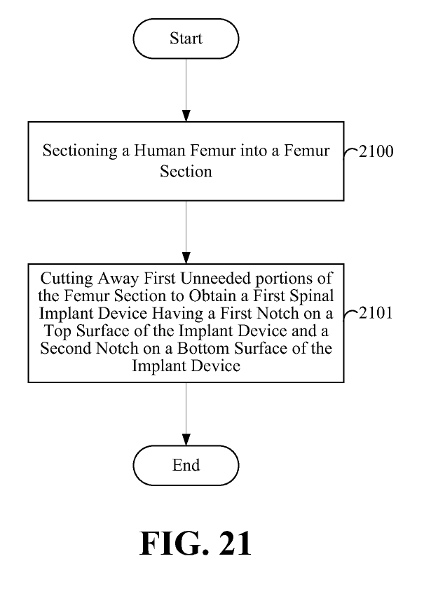

FIG. 11 is a cross-sectional view of the device 6 taken along line C-C in FIG. 10. As described hereinabove, the device 6 has the front surface 41 that is arch-shaped and which is formed from the outer wall 2 (FIG. 6) of the femur section 22 (FIG. 6). Additionally, the device 6 has a back surface 42 that has an arch portion 43. The arch portion 43 is formed by the inner wall 3 (FIG. 6) of the femur section 22. The remaining back surface 42 is formed when the unneeded portion 7 (FIG. 8) of the femur 22 (FIG. 8) is cut away from the device 6. In another embodiment, the front surface 41 and the arch portion 43 of the back surface 42 only approximate the outer wall 2 and the inner wall 3, respectively.

FIG. 12 is a side view of the device 6. The side view of the device 6 depicts the notch 33 that is formed in the side 31. In addition, the side view depicts the arched characteristic of the front surface 41. Further, the device 6 comprises a machined surface 10 that is cut so as to minimize sharp corners on the implant device 6.

FIG. 13 is a perspective view of the device 6. The device 6 comprises on the top side 34 the notch 36 for receiving a spinous process, which is described further with reference to FIG. 18. In addition, the device 6 comprises the sides 30 and 31 having notches 32 and 33, respectively, for receiving an insertion tool for inserting the implant device 6 into a functional spinal unit.

As indicated hereinabove, the front surface 41 is formed by the outer wall 2 (FIG. 6) of the femur section 22 (FIG. 6). Additionally, the back surface 42 is partially formed by the inner wall 3 (FIG. 6) of the femur section 22.

FIG. 14 is a perspective view of the device 6. The device 6 comprises the front surface 41 and the back surface 42. The back surface 42 is partially made up of the arched portion 43 that is formed by the inner wall 3 (FIG. 6) of the femur section 22 (FIG. 6). In addition, the back surface 43 comprises machined surface 11 that are formed when the unneeded portion 7 (FIG. 8) is cut away from the implant device 6.

The device 6 further has the sides 30 and 31. Side 30 has the notch 32, and side 31 has the notch 33. The notches 32 and 33 are for receiving an insertion tool.

The device further has the top side 34 and bottom side 35. The top side 34 comprises the notch 36 for receiving a spinous process, described further with reference to FIG. 18. Further, the bottom side 35 comprises the notch 37 for receiving a spinous process, described further with reference to FIG. 18

FIG. 15 is a side view of a functional spinal unit 60. The functional spinal unit 60 comprises a superior vertebral body 12 and corresponding spinous process 15. In addition, the functional spinal unit 60 comprises an inferior vertebral body 13 and corresponding spinous process 16. Further, a disc 14 is interposed between the superior vertebral body 12 and the inferior vertebral body 13. There is also an interspinous space 17 in which the implant device 6 (FIG. 10) can be inserted.

FIG. 16 is a posterior view of the functional spinal unit 60. As described hereinabove, the functional spinal unit 60 comprises the superior vertebral body 12 and corresponding spinous process 15. In addition, the functional spinal unit comprises the inferior vertebral body 13 and corresponding spinous process 16. Between the spinous process 15 and the spinous process 16 is the interspinous space 17 in which the implant device 6 can be inserted.

FIG. 17 is a lateral view of the functional spinal unit 60 having the implant device 6 inserted therein. In this regard, the implant device 6 is seated between the spinous process 15 and the spinous process 16. Seating the implant device in the interspinous space 17 in such a manner separates the superior vertebral body 12 from the inferior vertebral body 13 so that the disc 14 is relieved from pinching that may occur due to an ailment.

FIG. 18 is a posterior view of a functional spinal unit 60. The functional spinal unit 60 comprises the superior vertebral body 12 and corresponding spinous process 15 and the inferior vertebral body 13 and corresponding spinous process 16. The implant device 6 is seated within the interspinous space 17 (FIG. 17) such that the notch 36 receives the spinous process 15 and the notch 37 receives the spinous process 16.

FIG. 19 is a perspective view of the functional spinal unit 60. The functional spinal unit 60 comprises the superior vertebral body 12 and corresponding spinous process 15. In addition, the functional spinal unit 60 comprises the inferior vertebral body 13 and corresponding spinous process 16. Between the superior vertebral body 12 and the inferior vertebral body 13 is the disc 14. Between the spinous process 14 and the spinous process 15 is the interspinous space 17.

FIG. 20 is a perspective view of a functional spinal unit 60 wherein the implant device 6 is inserted. The implant device 6 is inserted such that the notch 36 receives the spinous process 15 and the notch 37 receives the spinous process 16.

FIG. 21 is a flowchart depicting a method of making the spinal implant device 6 depicted in FIG. 13.

The first step in the method is sectioning a human femur 1 (FIG. 1) into a femur section 22 (FIG. 2). The second step in the method is cutting away unneeded portions 7 (FIG. 8) of the human femur section 22 to obtain a spinal implant device 6 (FIG. 13) having a notch 36 (FIG. 10) on a top surface 34 (FIG. 10) of the implant device 6 and a notch 37 (FIG. 10) on a bottom surface 35 (FIG. 10) of the implant device.

Furthermore, the human femur section 22 can be used to create more than one spinal implant device 6. In this regard, on one side of the human femur section 22 a first spinal implant device 6 can be cut away from the unneeded portion 7. Thereafter, a second spinal implant device 6 can be cut out of the unneeded portion 7, thereby creating two spinal implant devices 6 out of the same femur section 22.

One skilled in the art will readily appreciate that the present disclosure is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the disclosure and are defined by the scope of the appended claims. Although the invention has been described in connection with specific preferred embodiments, it should be understood that the device as claimed should not be unduly limited to such specific embodiments. Indeed, various modifications of the described modes for carrying out the device which are obvious to those skilled in the art are intended to be within the scope of the following claims.

1. An implant device, comprising
a main body having a top surface and a bottom surface; and
a first notch formed in the top surface for receiving a first spinous process; and
a second notch formed in the bottom surface for receiving a second spinous process.

2. The implant device of claim 1, wherein the main body is substantially arch-shaped.

3. The implant device of claim 1, wherein the first notch comprises tapered walls.

4. The implant device of claim 3, wherein the second notch comprises tapered walls.

5. The implant device of claim 1, wherein the main body is made from a human femur section.

6. The implant device of claim 5, wherein a front surface of the main body approximates the outer wall of the human femur section.

7. The implant device of claim 5, wherein a front surface of the main body is the outer wall of the human femur section.

8. The implant device of claim 5, wherein a back surface of the main body approximates the inner wall of the human femur section.

9. The implant device of claim 5, wherein a back surface of the main body is the inner wall of the human femur section.

10. The implant device of claim 1, wherein the main body is made from human allograft bone.

11. The implant device of claim 1, further comprising a first and second side surface, wherein the first side surface has a third notch and the second side surface has a fourth notch, the third and forth notches for receiving an insertion tool.

12. The implant device of claim 1, wherein the first notch and the second notch have side walls and the side walls of the first notch are not parallel and the side walls of the second notch are not parallel.

13. The implant device of claim 12, wherein the first and second notches have a dimensioned width at the bottom of the first and second notches that is greater than a dimensioned width at the top of the first and second notches.

14. The implant device of claim 12, wherein the first and second notches have a dimensioned width at the bottom of the first and second notches that is less than a dimensioned width at the top of the first and second notches.

15. The implant device of claim 12, wherein the side walls are curved.

16. The implant device of claim 1, wherein the main body is composed of a synthetic implant grade polymer.

17. The implant device of claim 16, wherein the synthetic implant grade polymer is PEEK.

18. The implant device of claim 16, wherein the synthetic implant grade polymer is Polyethylene.

19. A method of manufacturing an implant device, comprising:
sectioning a human femur into a femur section;
cutting away first unneeded portions of the femur section to obtain a first spinal implant device having a first notch on a top surface of the first implant device and a second notch on a bottom surface of the first implant device.

20. The method of claim 19, further comprising the step of cutting away second unneeded portions of the human femur to obtain the first spinal implant device having a third notch on a first side surface and a fourth notch on a second side surface.

21. The method of claim 19, further comprising the step of cutting away second unneeded portions of the human femur to obtain a second spinal implant device.
